(19) United States
(12) Patent Application Publication
(10) Pub. No.: US 2019/0116820 A1
(43) Pub. Date: Apr. 25, 2019

(54) FERMENTED MILK PRODUCTION METHOD, METHOD FOR SHORTENING FERMENTATION TIME IN FERMENTED MILK PRODUCTION, METHOD FOR SUPPRESSING INCREASED SOURNESS IN FERMENTED MILK

(71) Applicant: MEIJI CO., LTD., Tokyo (JP)

(72) Inventors: Keisuke FURUICHI, Odawara-shi, Kanagawa (JP); Saori TAKAHASHI, Odawara-shi, Kanagawa (JP); Mizue SAITOU, Odawara-shi, Kanagawa (JP)

(73) Assignee: MEIJI CO., LTD., Tokyo (JP)

(21) Appl. No.: 16/093,599

(22) PCT Filed: Apr. 13, 2017
(86) PCT No.: PCT/JP2017/015057

§ 371 Date: Oct. 12, 2018

(30) Foreign Application Priority Data
Apr. 14, 2016 (JP) 2016-081190

(51) Int. Cl. A23C 9/127; C12N 1/20

(52) U.S. Cl. CPC A23C 9/127; C12N 1/20

(57) ABSTRACT

An object of the method is to simultaneously attain, by a simple method, shortening of the fermentation time (promotion of fermentation) at the time of fermented milk production and suppression of an increase in sourness during refrigerated transport and/or refrigerated storage of fermented milk. A method for producing fermented milk, a method for shortening the fermentation time at the time of fermented milk production, and a method for suppressing an increase in sourness, each including blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk, are also provided.

### TECHNICAL FIELD

The present invention relates to a method for producing fermented milk, a method for shortening the fermentation time in fermented milk production, and a method for suppressing an increase in sourness of fermented milk.

### BACKGROUND ART

The fermentation time is one of significant constraint conditions in fermented milk production. For example, if the fermentation time can be shortened, the production capability can be improved, for example, without enhancement of production facilities, with the result that the production cost can be effectively reduced. At this time, when a substance for promoting the growth of lactic acid bacterium (such as an enzyme decomposition product of milk protein) is blended into raw material milk in fermented milk production, the fermentation time can be shortened. However, there is a possibility that the resultant fermented milk can have an uncomfortable flavor as fermented milk due to the flavor and the like of the substance blended therein. Further, if the fermentation time is merely shortened in fermented milk production, there is a possibility that the fermentation of the fermented milk can be promoted as it is, for example, during refrigerated transport and/or refrigerated storage thereof, thereby causing an excessive increase in sourness and an uncomfortable flavor as fermented milk.

As techniques concerning the action of promoting the fermentation of fermented milk (such as yogurt), the following Patent Documents 1 to 8 can be exemplified. Patented Document 1 describes “lactic acid bacterium-containing fermented food product and method for producing the same” involving the use of guava leaf extract as an active ingredient; Patent Document 2 describes “proliferation promoter for bifidobacterium and lactobacillus” involving the use of sweet potato shochu (distilled liquor) lees as an active ingredient; Patent Document 3 describes “enhancer of proliferation of lactic acid bacterium, and agent for improvement in survivability of lactic acid bacterium” involving the use of acidic buttermilk containing a dead bacterial cell as an active ingredient; Patent Document 4 describes “growth promoter for lactic acid bacteria and food fermented by the lactic acid bacteria” involving the use of cis-vaccenic acid as an active ingredient; Patent Document 5 describes “fermentation promoter of lactic acid bacterium” involving the use of cocoa mass as an active ingredient; Patent Document 6 describes “growth promoter for lactic acid bacterium” involving the use, as an active ingredient, of crude pyroligneous acid obtained by dry distillation of wood; Patent Document 7 describes “substance for promoting proliferation of anaerobic bacterium” involving the use of calcium phosphate as an active ingredient; and Patent Document 8 describes “method for cultivating lactobacillus and food and drink” involving the use of ginger extract, tea extract and/or Welsh onion extract as an active ingredient/active ingredients.

Among Patented Documents 1 to 8, Patented Document 3 alone refers to the flavor of fermented milk. However, Patent Document 3 refers to the flavor of fermented milk, but merely describes: “. . . can enhance growth of the lactic acid bacterium and improve survivability of the lactic acid bacterium during storage without any adverse effect on the flavor and the production cost of the product.” This patent document does not refer to the action of improving the flavor of fermented milk or the action of suppressing an increase in sourness of fermented milk.

As techniques concerning fermented milk production using propionic acid bacteria, the “method for improving survivability of bifidus” in Patent Document 9 and the “agent and the method for improving survivability of lactic acid bacterium, and food composition” in Patent Document 10 can be exemplified.

Patent Document 9 describes, in Examples: “30 g of a product obtained by inoculating Propionibacterium freudenreichii IFO 12424, culturing the bacterium at 37° C. for 5 days and thermally treating the culture at 90° C. for 10 minutes was inoculated, and fermented at 43° C. for 3.5 hours.” In this patent document, propionic acid bacteria are killed by heat treatment before use, and a live bacterial cell of propionic acid bacteria is not concentrated. Therefore, the action of shortening the fermentation time, the action of improving the flavor of fermented milk and the action of suppressing an increase in sourness of fermented milk, by virtue of a live bacterial cell of propionic acid bacteria, are not sufficiently exerted.

Patent Document 10 describes, in Examples: “. . . 0.5% by weight of a fermentation product of propionic acid bacteria concentrated until the concentration of 1,4-dihydroxy-2-naphthoic acid (DHNA) reached 100 μg/ml, were simultaneously added to and mixed with the yogurt material and then fermented at 43° C. until the concentration of lactic acid reached 0.75%, to prepare yogurt,” and further describes, regarding the actually used fermentation product of propionic acid bacteria: “As a result, a culture (fermentation product of propionic acid bacteria) containing DHNA at a concentration of 52 μg/mL was obtained.” That is, since the concentration rate of the fermentation product of propionic acid bacteria is about twice, the action of shortening the fermentation time in fermented milk production, the action of improving the flavor of fermented milk and the action of suppressing an increase in sourness of fermented milk, by virtue of a live bacterial cell of propionic acid bacteria, are not sufficiently exerted.

### REFERENCE LIST

### Patent Documents

Patented Document 1: JP 2010-119305 A
Patented Document 2: JP 2009-125055 A
Patented Document 3: JP 2008-5811 A
Patented Document 4: JP 2006-262778 A
Patented Document 5: JP 2006-223244 A
Patented Document 6: JP 2005-318856 A
Patented Document 7: JP 2005-130804 A
Patented Document 8: JP 2001-190272 A
Patented Document 9: JP H7-227207 A
Patented Document 10: WO 2009/069498

### SUMMARY OF THE INVENTION

### Problem to be Solved by the Invention

As presented in the prior art, it is not easy to simultaneously attain (1) shortening of the fermentation time (promotion of fermentation) in fermented milk production and (2) suppression of an increase in sourness during refrigerated transport and/or refrigerated storage of fermented milk, by a simple method.

However, if the items (1) and (2) can be simultaneously attained by a simple method, great benefits can be provided in the commercial development involving large-scale fermented milk production.

An object of the present invention is to simultaneously attain the items (1) and (2) by a simple method.

### Means for Solving the Problem

The present invention is intended to attain this object by the following inventions.
[1] A method for producing fermented milk, comprising blending (adding) a predetermined amount of a live bacterial cell of propionic acid bacteria into raw material milk or a fermented milk substrate immediately before or immediately after blending (addition) of a lactic acid bacterium starter into the raw material milk, or simultaneously with blending (addition) of the lactic acid bacterium starter into the raw material milk.
[2] The method for producing fermented milk according to [1], wherein a culture liquid of propionic acid bacteria at a range of 0.0001 to 0.25% by mass is blended as the live bacterial cell of propionic acid bacteria into the raw material milk or fermented milk substrate.
[3] The method for producing fermented milk according to [1] or [2], wherein a culture liquid of propionic acid bacteria concentrated to 10 times or more is blended as the live bacterial cell of propionic acid bacteria into the raw material milk or fermented milk substrate.
[4] The method for producing fermented milk according to any one of [1] to [3], wherein the live bacterial cell of propionic acid bacteria at a concentration of 6×10⁶ cfu/mL or more is blended into the raw material milk or fermented milk substrate.
[5] A method for shortening the fermentation time in fermented milk production, comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.
[6] The method for shortening the fermentation time according to [5], wherein a culture liquid of propionic acid bacteria at a range of 0.0001 to 0.25% by mass is blended as the live bacterial cell of propionic acid bacteria into the raw material milk or fermented milk substrate.
[7] The method for shortening the fermentation time according to [5] or [6], wherein a culture liquid of propionic acid bacteria concentrated to 10 times or more is blended as the live bacterial cell of propionic acid bacteria into the raw material milk or fermented milk substrate.
[8] The method for shortening the fermentation time according to any one of [5] to [7], wherein the live bacterial cell of propionic acid bacteria at a concentration of 6×10⁶ cfu/mL or more is blended into the raw material milk or fermented milk substrate.
[9] A method for suppressing an increase in sourness (during refrigerated transport and/or refrigerated storage) of fermented milk, comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.
[10] The method for suppressing an increase in sourness (during refrigerated transport and/or refrigerated storage) of fermented milk according to [9], wherein a culture liquid of propionic acid bacteria at a range of 0.0001 to 0.25% by mass is blended as the live bacterial cell of propionic acid bacteria into the raw material milk or fermented milk substrate.
[11] The method for suppressing an increase in sourness (during refrigerated transport and/or refrigerated storage) of fermented milk according to [9] or [10], wherein a culture liquid of propionic acid bacteria concentrated to 10 times or more is blended as the live bacterial cell of propionic acid bacteria into the raw material milk or fermented milk substrate.
[12] The method for suppressing an increase in sourness (during refrigerated transport and/or refrigerated storage) of fermented milk according to any one of [9] to [11], wherein the live bacterial cell of propionic acid bacteria at a concentration of 6×10⁶ cfu/mL or more is blended into the raw material milk or fermented milk substrate.

In addition, the phrase “culture liquid of propionic acid bacteria concentrated to 10 times or more” in the above [3], [7] and [11] means, for example, that the live bacterial cell in the culture liquid of propionic acid bacteria is concentrated 10 times or more.

### Effect of the Invention

According to the present invention, (1) the shortening of the fermentation time (promotion of fermentation) in (at the time of) fermented milk production and (2) the suppression of an increase in sourness (during refrigerated transport and/or refrigerated. storage) of fermented milk can be simultaneously attained by a simple method.

### BRIEF DESCRIPTION OF THE DRAWINGS

FIG. 1 is a graph showing the relationship between the kind (strain) of propionic acid bacteria to be blended (added) into a fermented milk substrate and the action of promoting fermentation in fermented milk production.

FIG. 2 is a graph showing the relationship between the initial blending (adding) concentration of propionic acid bacteria (live bacterial cell) into the fermented milk substrate and the action of promoting fermentation in fermented milk production.

FIG. 3 is a graph showing the relationship between the blending (adding) concentration of a frozen concentrated bacterial liquid (culture liquid) into the fermented milk substrate and the action of promoting fermentation in fermented milk production.

FIG. 4 is a graph showing the relationship between the blending (adding) concentration of a frozen bacterial liquid (intact culture liquid) into the fermented milk substrate or the blending concentration of the frozen concentrated bacterial liquid and the concentration of lactic acid during refrigerated storage of fermented milk.

FIG. 5 is a graph showing the relationship between the kind (strain) of propionic acid bacteria to be blended (added) into the fermented milk substrate or the kind (strain) of a lactic acid bacterium starter and the action of promoting fermentation in fermented milk production.

FIG. 6 is a graph showing the relationship (degree of reduction in pH of a product refrigerated and stored for 7 days from immediately after fermentation) between the kind (strain) of propionic acid bacteria to be blended (added) into the fermented milk substrate or the kind (strain) of the lactic acid bacterium starter and the action of suppressing a reduction in pH of fermented milk.

FIG. 7 is a graph the relationship (degree of reduction in pH of a product refrigerated and stored for 14 days from immediately after fermentation) between the kind (strain) of propionic acid bacteria to be blended (added) into the fermented milk substrate or the kind (strain) of the lactic acid bacterium starter and the action of suppressing a reduction in pH of fermented milk.

### DETAILED DESCRIPTION OF THE INVENTION

On embodiment of the present invention involves a method for producing fermented milk, a method for shortening the fermentation time in fermented milk production, and a method for suppressing an increase in sourness of fermented milk, each comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.

Here, the method for suppressing an increase in sourness of fermented milk is, for example, a method for suppressing an increase in sourness during refrigerated transport and/or refrigerated storage of fermented milk.

In the present invention, the treatment of combining a predetermined amount of a live bacterial cell of propionic acid bacteria with raw material milk or a fermented milk substrate, the treatment of adding a predetermined amount of a live bacterial cell of propionic acid bacteria to raw material milk or a fermented milk substrate, the treatment of mixing a predetermined amount of a live bacterial cell of propionic acid bacteria and raw material milk or a fermented milk substrate, and the like are expressed collectively as “blending a predetermined amount of a live bacterial cell of propionic acid bacteria into raw material milk or a fermented milk substrate.” All the treatments expressed. using the terms “blending,” “blending (adding),” “adding,” “adding (blending),” “adding (mixing)” and “mixing” used herein are included in the concept of “blending” as defined above.

As a result of earnest studies on the conditions for producing fermented milk and the like, the present inventors have found that (1) the shortening of the fermentation time (promotion of fermentation) in (at the time of) fermented milk production and (2) the suppression of an increase in sourness (during refrigerated transport and/or refrigerated storage) of fermented milk can be simultaneously attained by blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk, and then have completed the present invention.

Since the fermentation time can be shortened according to the present invention, the producing steps can be effectively rationalized. The present invention makes it possible to improve the production capability, to effectively reduce the production cost, and to provide great benefits in the commercial development involving large-scale fermented milk production, without enhancement of production facilities.

When the present inventors blended each of various live bacterial cells of propionic acid bacteria in a predetermined amount, for example, in a predetermined concentration, into raw material milk or a fermented milk substrate and fermented them, the fermentation time was shortened, and a clear action of promoting fermentation could be confirmed. Specifically, the fermentation time was shortened by disinfecting raw material milk (yogurt mix) and blending (adding) a common lactic acid bacterium starter thereinto to prepare a fermented milk substrate (yogurt base), then blending (adding) each of various live bacterial cells of propionic acid bacteria, in a predetermined concentration, into the fermented milk substrate and fermenting them, and a clear action of promoting fermentation could also be confirmed.

In the present invention, the “raw material milk” means a fluid in a liquid form, a gel form or the like, containing a nutritional component such as a lipid, a protein, a saccharide, a mineral or a vitamin, which fluid has been prepared to produce fermented milk. At this lime, the above fluid before disinfection or the above fluid after disinfection may be employed, but the above fluid after disinfection is preferred, for example, from the viewpoint of hygienic production of fermented milk on a commercial scale.

Also, in the present invention, the “fermented milk substrate” means a fluid obtained by blending (adding) a lactic acid bacterium starter into the raw material milk described above.

In the above-described embodiment of the present invention, one embodiment in which a predetermined amount of a live bacterial cell of propionic acid bacteria is blended into raw material milk or a fermented milk substrate involves blending the live bacterial cell of propionic acid bacteria into the raw material milk or fermented milk substrate using less than 0.5 parts by mass (% by mass) of a culture liquid of propionic acid bacteria. This is an embodiment directed to a method for producing fermented milk, a method for suppressing the fermentation time (method for promoting fermentation) in fermented milk production, and a method for suppressing an increase in sourness of fermented milk, each comprising such procedures.

In the above-described embodiment of the present invention, another embodiment in which a predetermined amount of a live bacterial cell of propionic acid bacteria is blended into raw material milk or a fermented Milk substrate involves concentrating (a live bacterial cell in) the culture liquid of propionic acid bacteria 10 times or more and blending the live bacterial cell of propionic acid bacteria into the raw material milk or fermented milk substrate. This is an embodiment directed to a method for producing fermented milk, a method for suppressing the fermentation time (method for promoting fermentation) in fermented milk production, and a method for suppressing an increase in sourness of fermented milk, each comprising such procedures.

In the above-described embodiment of the present invention, still another embodiment in which a predetermined amount of a live bacterial cell of propionic acid bacteria is blended into raw material milk or a fermented milk substrate involves blending the live bacterial cell of propionic acid bacteria, at a concentration of 6×10⁶ cfu/mL or more, into the raw material milk or fermented milk substrate. This is an embodiment directed to a method for producing fermented milk, a method for suppressing the fermentation time (method for promoting fermentation) in fermented milk production, and a method for suppressing an increase in sourness of fermented milk, each comprising such procedures.

The above-described embodiments involving blending the live bacterial cell of propionic acid bacteria into the raw material milk or fermented milk substrate using less than 0.5 parts by mass (% by mass) of a culture liquid of propionic acid bacteria; involving concentrating (a live bacterial cell in) the culture liquid of propionic acid bacteria 10 times or more and blending the live bacterial cell of propionic acid bacteria into the raw material milk or fermented milk substrate; and involving blending the live bacterial cell of propionic acid bacteria, at a concentration of 6×10⁶ cfu/mL or more, into the raw material milk or fermented milk substrate, can each be forms to be embodied singly. Alternatively, two or more of these embodiments can be arbitrarily combined.

In all of the embodiments, it is possible to produce fermented milk, to realize the shortening of the fermentation time (promotion of fermentation) in fermented milk production, and to suppress an increase in sourness of fermented milk (for example, an increase in sourness during refrigerated transport and/or refrigerated storage of fermented milk).

Hence, (1) the shortening of the fermentation time (promotion of fermentation) in (at the time of) fermented milk production and (2) the suppression of an increase in sourness (during refrigerated transport and/or refrigerated storage) of fermented milk can be simultaneously attained by a simple method.

Since the fermented milk to be produced by the above-described embodiments has an improved flavor, these embodiments are also embodiments directed to a method for improving the flavor of fermented milk. According to each of the above-described embodiments, it is possible to simultaneously attain (1) the shortening of the fermentation time (promotion of fermentation) in (at the time of) fermented milk production, (2) the suppression of an increase in sourness (during refrigerated transport and/or refrigerated storage) of fermented milk, and (3) the improvement of the flavor of fermented milk, by a simple method.

When the present inventors confirmed the flavor of fermented milk in each of the above-described embodiments according to the present invention, the flavor of the fermented milk could be improved, so that fermented milk having an improved flavor could be obtained. The flavor of the fermented milk could not be recognized as being good when the culture liquid of propionic acid bacteria was not added to the fermented milk substrate or (the live bacterial cell in) the culture liquid of propionic acid bacteria was concentrated less than 10 times and added to the fermented milk substrate. The reason for this was considered to be because the flavor derived from the culture liquid of propionic acid bacteria affected the flavor of the fermented milk. For example, the flavor of the fermented milk could not be recognized as being good when (the live bacterial cell in) the culture liquid of propionic acid bacteria was concentrated 5 times and added to the fermented milk substrate.

According to the studies by the present inventors, when (the live bacterial cell in) the culture liquid of propionic acid bacteria was concentrated 10 times or more and blended into the raw material milk or fermented milk substrate, the action of promoting fermentation (action of shortening the fermentation time) could be exerted.

Also when the live bacterial cell of propionic acid bacteria was blended, at a concentration of 6×10⁶ cfu/mL or more, into the raw material milk or fermented milk substrate, the action of promoting fermentation (action of shortening the fermentation time) could be exerted.

Also when (the live bacterial cell in) the culture liquid of propionic acid bacteria was concentrated 10 times or more and the live bacterial cell of propionic acid bacteria was blended at a concentration of 6×10⁶ cfu/mL or more, into the raw material milk or fermented milk substrate, the action of promoting fermentation (action of shortening the fermentation time) could be exerted.

When the present inventors confirmed an increase in sourness during refrigerated storage of fermented milk in each of the above-described embodiments according to the present invention, fermented milk in which the increase in sourness was suppressed and the flavor hardly changed with time, as compared with the case of using a conventional fermented milk substrate containing no live bacterial cell of propionic acid bacteria, could be obtained, in all of the embodiments involving blending the live bacterial cell of propionic acid bacteria into the raw material milk or fermented milk substrate using less than 0.5 parts by mass (% by mass) of a culture liquid of propionic acid bacteria; involving concentrating (a live bacterial cell in) the culture liquid of propionic acid bacteria 10 times or more and blending the live bacterial cell of propionic acid bacteria into the raw material milk or fermented milk substrate; and involving blending the live bacterial cell of propionic acid bacteria, at a concentration of 6×10⁶ cfu/mL or more, into the raw material milk or fermented milk substrate.

Briefly, it could not be recognized that the increase in sourness of fermented milk was sufficiently suppressed when (the live bacterial cell in) the culture liquid of propionic acid bacteria was not added to the fermented milk substrate, (the live bacterial cell in) the culture liquid of propionic acid bacteria was concentrated less than 10 times and added to the fermented milk substrate, or the live bacterial cell of propionic acid bacteria was added at a concentration of less than 6×10⁶ cfu/mL to the fermented milk substrate. For example, when (the live bacterial cell in) the culture liquid of propionic acid bacteria was not added to the fermented milk substrate, the increase in sourness during refrigerated transport and/or refrigerated storage of fermented milk was not sufficiently suppressed.

On the other hand, the shortening of the fermentation time (promotion of fermentation) in fermented milk production and the suppression of an increase in sourness of fermented milk (an increase in sourness during refrigerated transport and/or refrigerated storage of fermented milk) could be realized in fermented milk production when a predetermined amount of a live bacterial cell of propionic acid bacteria was blended into raw material milk or a fermented milk substrate, for example, when a live bacterial cell of propionic acid bacteria was blended into raw material milk or a fermented milk substrate using less than 0.5 parts by mass (% by mass) of a culture liquid of propionic acid bacteria; (a live bacterial cell in) a culture liquid of propionic acid bacteria was concentrated 10 times or more and a live bacterial cell of propionic acid bacteria was blended into raw material milk or a fermented milk substrate; or a live bacterial cell of propionic acid bacteria was blended, at a concentration of 6×10⁶ cfu/mL or more, into raw material milk or a fermented milk substrate, or when a plurality of blending forms, among these blending forms, were arbitrarily combined. Fermented milk with an improved flavor could also be produced.

In the present invention, as the embodiment in which a predetermined amount of a live bacterial cell of propionic acid bacteria is blended into raw material milk or a fermented milk substrate, when a live bacterial cell of propionic acid bacteria was blended into raw material milk or a fermented milk substrate using less than 0.5 parts by mass (% by mass) of a culture liquid of propionic acid bacteria, the blending (adding) concentration of (the live bacterial cell in) the culture liquid of propionic acid bacteria into the raw material milk or fermented milk substrate is preferably 0.4 parts by mass (% by mass) or less, more preferably 0.3 parts by mass (% by mass) or less, more preferably 0.25 parts by mass (% by mass) or less, more preferably 0.2 parts by mass (% by mass) or less, more preferably 0.15 parts by mass (% by mass) or less, more preferably 0.1 parts by mass (% by mass) or less, more preferably 0.05 parts by mass (% by mass) or less, further preferably 0.03 parts by mass (% by mass) or less from the viewpoint of promoting the fermentation of fermented milk, improving the flavor of fermented milk, and suppressing an increase in sourness of fermented milk. At this time, the blending concentration of the culture liquid of propionic acid bacteria into the raw material milk or fermented milk substrate is not particularly limited so long as the concentration is 0.4 parts by mass (% by mass) or less, but is preferably 0.0001 parts by mass (% by mass) or more, more preferably 0.0005 parts by mass (% by mass) or more, more preferably 0.0008 parts by mass (% by mass) or more, further preferably 0.001 parts by mass (% by mass) or more, from the viewpoint of suppressing the labor and time required to concentrate the culture liquid of propionic acid bacteria.

In the present invention, as the embodiments in which a predetermined amount of a live bacterial cell of propionic acid bacteria is blended into raw material milk or a fermented milk substrate, when a culture liquid of propionic acid bacteria is concentrated 10 times or more and blended into raw material milk or a fermented milk substrate, i.e., a live bacterial cell in the culture liquid of propionic acid bacteria is concentrated 10 times or more and blended into the raw material milk or fermented milk substrate, the concentration rate of (the live bacterial cell in) the culture liquid of propionic acid bacteria is preferably 10 times or more, more preferably 12 times or more, more preferably 15 times or more, more preferably 17 times or more, further preferably 20 times or more, from the viewpoint of promoting the fermentation of fermented milk, improving the flavor of fermented milk, and suppressing an increase in sourness of fermented milk. At this time, the concentration rate of the culture liquid of propionic acid bacteria is not particularly limited so long as the concentration rate is 10 times or more, but is preferably 50 times or less, more preferably 40 times or less, more preferably 30 times or less, further preferably 25 times or less, from the viewpoint of the labor and time required to concentrate the culture liquid of propionic acid bacteria.

In the present invention, as the embodiment in which a predetermined amount of a live bacterial cell of propionic acid bacteria is blended into raw material milk or a fermented milk substrate, when a live bacterial cell of propionic acid bacteria is blended at a concentration of 6×10⁶ cfu/mL or more into raw material milk or a fermented milk substrate, the blending concentration of the live bacterial cell of propionic acid bacteria is preferably 6×10⁶ cfu/mL or more, more preferably 1×10⁷ cfu/mL or more, more preferably 3×10⁷ cfu/mL or more, more preferably 6×10⁷ cfu/mL or more, more preferably 1×10⁸ cfu/mL or more, further preferably 3×10⁸ cfu/mL or more from the viewpoint of promoting the fermentation of fermented milk, improving the flavor of fermented milk, and suppressing an increase in sourness of fermented milk. At this time, the blending concentration of the live bacterial cell of propionic acid bacteria is not particularly limited so long as the concentration is 6×10⁶ cfu/mL or more, but is preferably 3×10⁹ cfu/mL or less, more preferably 1×10⁹ cfu/mL or less, further preferably 6×10⁸ cfu/mL or less from the viewpoint of suppressing the numerical quantity of the live bacterial cell of propionic acid bacteria to be used and the cost.

In the embodiments according to the present invention, the time until the acidity reaches 0.7% (a guide of the end point of fermentation) during fermentation is shortened preferably by 10 minutes or more, more preferably by 20 minutes or more, more preferably by 30 minutes or more, more preferably by 40 minutes or more, more preferably by 50 minutes or more, further preferably by 60 minutes or more, as compared with conventional products containing no (live bacterial cell in) culture liquid of propionic acid bacteria.

When the present inventors used various kinds of propionic acid bacteria to study the effects and actions of the present invention, whatever kind of propionic acid bacteria are used, the action of promoting fermentation, the action of improving the flavor of fermented milk, and the action of suppressing an increase in sourness of fermented milk, as described above, were exerted by blending the live bacterial cell of propionic acid bacteria in a predetermined concentration into the raw material milk or fermented milk substrate.

Therefore, it was considered that, for all of propionic acid bacteria without limitation on the kind of propionic acid bacteria, the action of promoting fermentation, the action of improving the flavor of fermented milk, and the action of suppressing an increase in sourness of fermented milk were exerted by blending a predetermined amount of the live bacterial cell of propionic acid bacteria into the raw material milk or fermented milk substrate.

When the present inventors used various kinds of lactic acid bacterium starters to study the effects and actions of the present invention, whatever kind of lactic acid bacterium starter is used, the action of promoting fermentation, the action of improving the flavor of fermented milk, and the action of suppressing an increase in sourness of fermented milk, as described above, were exerted by blending a predetermined amount of the live bacterial cell of propionic acid bacteria into the raw material milk or fermented milk substrate.

Therefore, it was considered that, for all of lactic acid bacterium starters without limitation on the kind of lactic acid bacterium starter, the action of promoting fermentation, the action of improving the flavor of fermented milk, and the action of suppressing an increase in sourness of fermented milk were exerted by blending a predetermined amount of the live bacterial cell of propionic acid bacteria into the raw material milk or fermented milk substrate.

Hereinafter, the present invention will be described in more detail by way of several Examples. The present invention is not limited to the above-described embodiments and the following Examples, and can be variously modified within the technical scope understood from the claims.

### EXAMPLES

### Example 1

Relationship between kind (strain) of propionic acid bacteria to be blended (added) into fermented milk substrate and action of promoting fermentation in fermented milk production

A culture liquid containing a live bacterial cell of propionic acid bacteria was prepared as follows.

Into 2350 g (94 parts by mass) of ultrapure water (Milli Q), 150 g (6 parts by mass) of skimmed milk powder (manufactured by Meiji Co., Ltd.) was blended (mixed), and 2.5 g (0.1 parts by mass) of a proteolytic enzyme (Amano A, manufactured by Amano Enzyme Inc.) was blended (mixed) thereinto. Then, an aqueous NaOH solution (4N) was used to adjust the pH to about 7.0. This solution was warmed and retained (47° C., 2 hours) while the pH thereof was adjusted to about 7.0. Thereafter, this solution was warmed and retained (47° C. 1 hour) without adjusting the pH thereof. Into this solution, 50 g (2 parts by mass) of lactose (manufactured by Leprino Foods Company) and 12.5 g (0.5 parts by mass) of yeast extract (Yeast extract, manufactured by Asahi Breweries, Ltd.) were blended (mixed), and the mixture was sterilized in an autoclave (121° C., 5 minutes) and then ice-cooled (5° C. or lower) to prepare a medium.

Into this medium, each of propionic acid bacteria (MEP1404201 strain, MEP1404202 strain, ET-3, MEP1404203 strain, MEP1404204 strain, MEP1404205 strain, MEP1404206 strain, MEP1404207 strain, and MEP1404208 strain) was added (mixed) in an amount of 1 part by mass and then statically cultured (30° C., 3 to 4 days) to prepare culture liquids containing live bacterial cells of various kinds of propionic acid bacteria.

These culture liquids were centrifuged to concentrate the live bacterial cells of various kinds of propionic acid bacteria 20 times. Then, the concentrates were frozen in liquid nitrogen (−80° C.) to prepare frozen concentrates (frozen concentrated bacterial liquids) containing the live bacterial cells of various kinds of propionic acid bacteria.

The characteristic features of the propionic acid bacteria MEP1404201 strain, MEP1404202 strain, ET-3, MEP1404203 strain, MEP1404204 strain, MEP1404205 strain, MEP1404206 strain, MEP1404207 strain, and MEP1404208 strain are indicated in Table 1.

The propionic acid bacteria ET-3 (Propionibacterium freudenreichii ET-3) is deposited with National Institute of Advanced Industrial Science and Technology Patent Microorganisms Depositary. The details for specifying the deposit are described below.
(1) Depositary institution name: National Institute of Advanced Industrial Science and Technology Patent Microorganisms Depositary
(2) Contact: Central 6, 1-1-1 Higashi, Tsukuba, Ibaraki 305-8566
(3) Accession No.: FERM BP-8115
(4) Indication for identification: ET-3
(5) Date of original deposit: Aug. 9, 2001
(6) Date of transfer to deposit based on Budapest Treaty: Jul. 11, 2002

TABLE 1
Names of 9 kinds of propionic acid bacteria
tested in Examples (strain names)

| Bacterial species | Strain name | Another name |
|---|---|---|
| P. freudenreichii subsp. Freudenreichii | ET-3 | |
| P. freudenreichii subsp. Globosum | MEP1404201 | (IFO 12424) |
| P. freudenreichii subsp. Globosum | MEP1404203 | (PS-1) |
| P. freudenreichii subsp. Shermanii | MEP1404204 | (PS-4) |
| P. freudenreichii subsp. Shermanii | MEP1404202 | (IFO 12391) |
| P. freudenreichii subsp. Shermanii | MEP1404205 | (ATCC9414T) |
| P. freudenreichii subsp. Shermanii | MEP1404206 | |
| P. freudenreichii subsp. Shermanii | MEP1404207 | |
| P. freudenreichii subsp. Shermanii | MEP1404208 | |

Mixed were 77.6 parts by mass of commercial cow's milk (manufactured by Meiji Co., Ltd.), 2.43 parts by mass of commercial skimmed milk powder (manufactured by Meiji Co., Ltd.) and 17.9 parts by mass of water were mixed, and the mixture was heated (disinfected) at 95° C. and for 5 minutes to prepare raw material milk.

Into this raw material milk, 2.0 parts by mass of a lactic acid bacterium starter (separated from commercial Meiji Bulgaria Yogurt LB81 (trade name)) and 0.06 parts by mass of the frozen concentrated bacterial liquids of 7 kinds of propionic acid bacteria, i.e., MEP1404202 strain, ET-3, MEP1404203 strain, MEP1404204 strain, MEP1404205 strain, MEP1404206 strain, and MEP1404208 strain, among the 9 kinds of propionic acid bacteria prepared in the above manner, were added (blended) to prepare fermented milk substrates containing the various kinds of propionic acid bacteria.

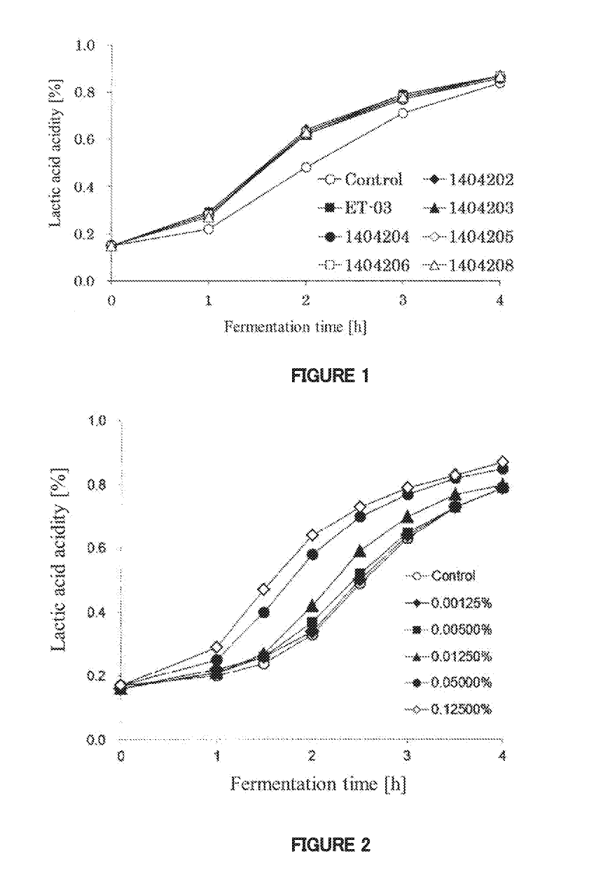

These fermented milk substrates were fermented (43° C.), and the changes in acidity of lactic acid with time are shown in FIG. 1. As a Control, a fermented milk substrate containing no propionic acid bacterium was fermented (43° C.), and the change in acidity of lactic acid with time is shown in FIG. 1.

From FIG. 1, in all of the fermented milk substrates containing the 7 kinds of propionic acid bacteria prepared in the above manner, fermentation was promoted, and the time until the acidity reached 0.7% (a guide of the end point of fermentation) was shortened by 30 minutes or more as compared with that of the Control (fermented milk substrate containing no propionic acid bacterium).

Briefly, the action of promoting fermentation was confirmed in the fermented milk substrates containing the 7 kinds of propionic acid bacteria prepared in the above manner.

### Example 2

Relationship between initial blending (adding) concentration of propionic acid bacteria (live bacterial cell) into fermented milk substrate and action of promoting fermentation in fermented milk production

Into a medium prepared in a similar manner as in Example 1, propionic acid bacteria ET-3 were added (mixed) in an amount of 1 part by mass and then statically cultured (30° C., 3 days) to prepare a culture liquid containing a live bacterial cell of propionic acid bacteria ET-3.

This culture liquid was centrifuged to concentrate the live bacterial cell of propionic acid bacteria ET-3 20 times. Then, the concentrate was frozen in liquid nitrogen (−80° C.) to prepare a frozen concentrate (frozen concentrated bacterial liquid) containing the live bacterial cell of propionic acid bacteria ET-3.

Mixed were 77.6 parts by mass of commercial cow's milk (manufactured by Meiji Co., Ltd.), 2.43 parts by mass of commercial skimmed milk powder (manufactured by Meiji Co., Ltd.) and 17.9 parts by mass of water were mixed, and the mixture was heated (disinfected) at 95° C. and for 5 minutes to prepare raw material milk.

Into this raw material milk, 2.0 parts by mass of a lactic acid bacterium starter (separated from commercial Meiji Bulgaria Yogurt LB81 (trade name)) and the frozen concentrated bacterial liquid of propionic acid bacteria ET-3 were mixed to prepare fermented milk substrates containing the propionic acid bacteria. The frozen concentrated bacterial liquid of propionic acid bacteria ET-3 was mixed in proportions of 0.00125 parts by mass, 0.005 parts by mass, 0.0125 parts by mass, 0.05 parts by mass, and 0.125 parts by mass, respectively, i.e., 5 kinds of frozen concentrated bacterial liquids were employed.

Since the concentration of a live bacterial cell of the propionic acid bacteria in this frozen concentrated bacterial liquid is 2.4×10¹¹ cfu/mL, the concentrations of the live bacterial cells of the propionic acid bacteria in these fermented milk substrates are 3×10⁶, 6×10⁶, 3×10⁷, 6×10⁷, and 3×10⁸ cfu/mL, respectively, according to the mixing proportions indicated above.

These fermented milk substrates were fermented (43° C.), and the changes in acidity of lactic acid with time are shown in FIG. 2. As a Control, a fermented milk substrate containing no propionic acid bacterium was fermented (43° C.), and the change in acidity of lactic acid with time is shown in FIG. 2.

From FIG. 2, in the fermented milk substrates containing the propionic acid bacteria, fermentation was promoted and the action of promoting fermentation was confirmed, when the blending concentration of the frozen concentrated bacterial liquid (20 times) was 0.005 parts by mass (% by mass) or more (the initial blending concentration of the propionic acid bacteria was 6×10⁶ cfu/mL or more).

### Example 3

Relationship between blending (adding) concentration of frozen concentrated bacterial liquid (culture liquid) into fermented milk substrate and action of promoting fermentation in fermented milk production

Into a medium prepared in a similar manner as in Example 1, propionic acid bacteria ET-3 were added (mixed) in an amount of 1 part by mass and then statically cultured (30° C., 3 days) to prepare a culture liquid containing a live bacterial cell of propionic acid bacteria ET-3.

On the other hand, this culture liquid was centrifuged to concentrate the live bacterial cell of propionic acid bacteria ET-3 5 times, 10 times, and 20 times, respectively. Then, the concentrates were frozen in liquid nitrogen (−80° C.) to prepare frozen concentrates (frozen concentrated bacterial liquids (5 times, 10 times, and 20 times)) containing the live bacterial cell of propionic acid bacteria ET-3.

Mixed were 77.6 parts by mass of commercial cow's milk (manufactured by Meiji Co., Ltd.), 2.43 parts by mass of commercial skimmed milk powder (manufactured by Meiji Co., Ltd.) and 17.9 parts by mass of water were mixed, and the mixture was heated (disinfected) at 95° C. and for 5 minutes to prepare raw material milk.

Into this raw material milk, 2.0 parts by mass of a lactic acid bacterium starter (separated from commercial Meiji Bulgaria Yogurt LB81 (trade name)) and the frozen bacterial liquid of propionic acid bacteria ET-3 were mixed to prepare fermented milk substrates containing the propionic acid bacteria. The frozen bacterial liquid of propionic acid bacteria ET-3 was adjusted as follows: frozen bacterial liquid (unconcentrated, 1 time): 2.5 parts by mass, frozen concentrated bacterial liquid (5 times): 0.5 parts by mass, frozen concentrated bacterial liquid (10 times): 0.25 parts by mass, and frozen concentrated bacterial liquid (20 times): 0.125 parts by mass of propionic acid bacteria ET-3, i.e., 4 kinds of frozen bacterial liquids of propionic acid bacteria ET-3 were employed.

Since the concentration of the live bacterial cell of the propionic acid bacteria in the culture liquid described above is 1.0×10¹⁰ cfu/mL, all the concentrations of the live bacterial cells of the propionic acid bacteria in these fermented milk substrates are 2.5×10⁸ cfu/mL.

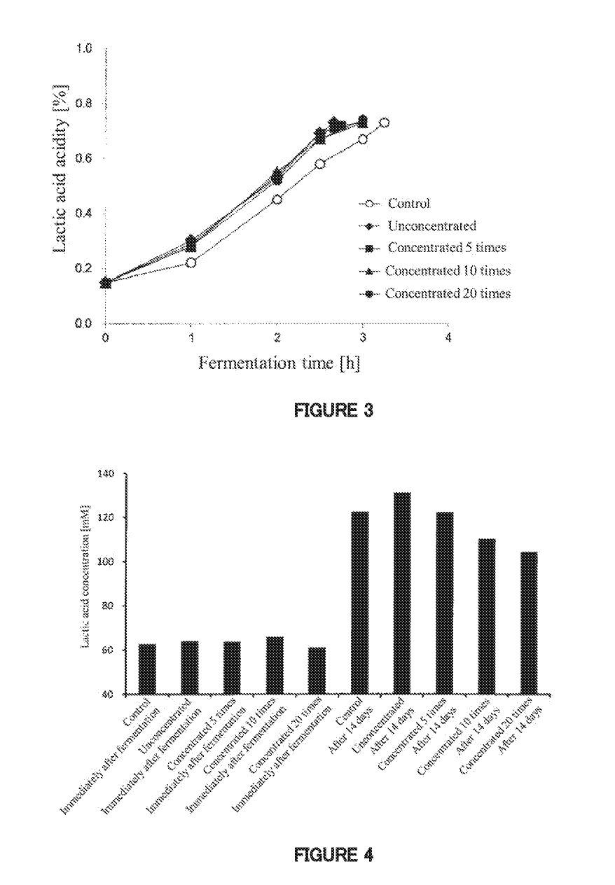

These fermented milk substrates were fermented (43° C.), and the changes in acidity of lactic acid with time are shown in FIG. 3. As a Control, a fermented milk substrate containing no propionic acid bacterium was fermented (43° C.), and the change in acidity of lactic acid with time is shown in FIG. 3.

From FIG. 3, in the fermented milk substrates containing the propionic acid bacteria, fermentation was promoted independently of the blending (adding) concentration of the frozen concentrated bacterial liquid (culture liquid), and the action of promoting fermentation was confirmed when the initial blending (adding) concentrations of the propionic acid bacteria (live bacterial cell) were equivalent.

From this, it was considered that the action of promoting fermentation was affected not by the chemical substances and nutritional components contained in the culture liquid or frozen concentrated bacterial liquid of the propionic acid bacteria, but by the propionic acid bacteria (live bacterial cell).

It was considered that the flavor of fermented milk might be deteriorated, for example, by the influences of the flavor of a culture liquid of propionic acid bacteria on the flavor of fermented milk when a frozen bacterial liquid (intact culture liquid) of propionic acid bacteria was blended into a fermented milk substrate and when a frozen concentrated bacterial liquid having a low concentration rate of propionic acid bacteria was blended thereinto.

So, the relationship between the blending (adding) concentration of the frozen bacterial liquid (intact culture liquid) into the fermented milk substrate or the blending concentration of the frozen concentrated bacterial liquid and the flavor of fermented milk was studied.

Specifically, various kinds of fermented milk were subjected to sensory evaluation (test) by 6 special panels based on the three items “Degree of sourness (1: strong; 2: rather strong; 3: ordinary; 4: rather weak; 5: weak),” “Yogurt-like flavor (1: undesirable; 2: rather undesirable; 3: ordinary; 4: rather desirable; 5: desirable),” and “Total deliciousness (1: undelicious; 2: rather undelicious; 3: ordinary; 4: rather delicious; 5: delicious), and “Comprehensive evaluation (average of the three items),” as indexes (criteria for evaluations. The results are indicated in Table 2.

TABLE 2
Relationship between blending concentration of culture liquid of propionic acid bacteria
and flavor of fermented milk

| Blending of propionic acid | Not | Blended (concentration rate of bacterial cell of | | | |
|---|---|---|---|---|---|
| bacteria | blended | propionic acid bacteria) | | | |
| Item of sensory evaluation | Control | (Unconcentrated) | (5 times) | (10 times) | (20 times) |
| Degree of sourness | 2 | 4 | 4 | 4 | 4 |
| Yogurt-like flavor | 4 | 2 | 2 | 5 | 5 |
| Total deliciousness | 4 | 1 | 1 | 5 | 5 |
| Comprehensive evaluation | 3.3 | 2.3 | 2.3 | 4.7 | 4.7 |

Degree of sourness (1: strong; 2: rather strong; 3: ordinary; 4: rather weak; 5: weak)
Yogurt-like flavor (1: undesirable; 2: rather undesirable; 3: ordinary; 4: rather desirable; 5: desirable)
Total deliciousness (1: undelicious; 2: rather undelicious; 3: ordinary; 4: rather delicious; 5: delicious
Comprehensive evaluation (average of the three items)

From Table 2, in the case of the fermented milk produced by blending 2.5 parts by mass (% by mass) of the frozen bacterial liquid (unconcentrated, 1 time) into the fermented milk substrate and the fermented milk produced by blending 0.5 parts by mass (% by mass) of the frozen concentrated bacterial liquid (5 times) into the fermented milk substrate, the “Degree of sourness” was weaker; the “Yogurt-like flavor” was more undesirable; the “Total deliciousness” was more undelicious; and the “Comprehensive evaluation” value was lower than those of the Control (fermented milk containing no propionic acid bacterium).

On the other hand, in the case of the fermented milk produced by blending 0.25 parts by mass (% by mass) of the frozen concentrated bacterial liquid (10 time) into the fermented milk substrate and the fermented milk produced by blending 0.125 parts by mass (% by mass) of the frozen concentrated bacterial liquid (20 times) into the fermented milk substrate, the “Degree of sourness” was weaker; the “Yogurt-like flavor” was more desirable; the “Total deliciousness” was more delicious; and the “Comprehensive evaluation” value was higher than those of the Control (fermented milk containing no propionic acid bacterium).

At this time, in the case of the fermented milk produced by blending 0.25 parts by mass (% by mass) of the frozen concentrated bacterial liquid (10 time) into the fermented milk substrate and the fermented milk produced by blending 0.125 parts by mass (% by mass) of the frozen concentrated bacterial liquid (2(i times) into the fermented milk substrate, the “mildness” and “richness,” which are important in flavor improvement, were enhanced.

That is, in the fermented milk containing the propionic acid bacteria, the flavor and texture were improved and the action of improving the flavor and the action of improving the texture were confirmed, when the blending concentration of the frozen concentrated bacterial liquid was 0.25 parts by mass (% by mass) or less (the concentration rate of the propionic acid bacteria (live bacterial cell) was 10 times or more).

As shown in FIG. 3, in the fermented milk substrates containing the propionic acid bacteria, fermentation was promoted independently of the blending (adding) concentration of the frozen concentrated bacterial liquid (culture liquid), and the action of promoting fermentation was confirmed when the initial blending (adding) concentrations of the propionic acid bacteria (live bacterial cell) were equivalent. At this time, it was considered that the blending of the propionic acid bacteria into the fermented milk substrate might promote fermentation and cause an excessive increase in lactic acid concentration of fermented milk (too strong sourness).

So, the relationship between the blending (adding) concentration of the frozen bacterial liquid (intact culture liquid) into the fermented milk substrate or the blending concentration of the frozen concentrated bacterial liquid and the lactic acid concentration during refrigerated storage of fermented milk was studied.

Specifically, for the various kinds of fermented milk prepared in the above manner, the lactic acid acidities of fresh products immediately after fermentation and the lactic acid concentrations of products refrigerated (5° C.) stored for 14 days immediately after fermentation were measured, and shown in FIG. 4.

From FIG. 4, it was confirmed that, in the case of the fermented milk produced by blending the frozen bacterial liquid or frozen concentrated bacterial liquid of propionic acid bacteria into the fermented milk substrate, the lactic acid concentration of the fresh product immediately after fermentation was equivalent to that of the Control (fermented milk containing no propionic acid bacterium), but that the lactic acid concentration of the product refrigerated stored for 14 days immediately after fermentation was different therefrom.

Actually, in the case of the fermented milk produced by blending 2.5 parts by mass (% by mass) of the frozen bacterial liquid (1 time) into the fermented milk substrate and the fermented milk produced by blending 0.5 parts by mass (% by mass) of the frozen concentrated bacterial liquid (5 times) into the fermented milk substrate, the lactic acid concentration of the product refrigerated stored for 14 days immediately after fermentation was increased to an equivalent level or higher, as compared with that of the Control (fermented milk containing no propionic acid bacterium).

On the other hand, in the case of the fermented milk produced by blending 0.25 parts by mass (% by mass) of the frozen concentrated bacterial liquid (10 time) into the fermented milk substrate and the fermented milk produced by blending 0.125 parts by mass (% by mass) of the frozen concentrated bacterial liquid (20 times) into the fermented milk substrate, the lactic acid concentration of the product refrigerated stored for 14 days immediately after fermentation was reduced, as compared with that of the Control (fermented milk containing no propionic acid bacterium).

Briefly, in the fermented milk containing propionic acid bacteria, the lactic acid concentration was reduced and the action of suppressing an increase in lactic acid concentration (action of reducing the sourness) was confirmed, when the blending concentration of the frozen concentrated bacterial liquid was 0.25 parts by mass (% by mass) or less (the concentration rate of the propionic acid bacteria (live bacterial cell) was 10 times or more).

At this time, in the fermented milk substrates containing the propionic acid bacteria, fermentation was promoted independently of the initial blending (adding) concentration of the propionic acid bacteria (live bacterial cell), and the action of reducing the lactic acid concentration (action of reducing the sourness) was confirmed when the blending (adding) concentrations of the frozen concentrated bacterial liquid (culture liquid) were different.

From this, it was considered that the action of reducing the lactic acid concentration and the action of reducing the sourness were not affected by the propionic acid bacteria (live bacterial cell), but that the action of suppressing an increase in lactic acid concentration (action of reducing the sourness) was affected by the chemical substances and nutritional components contained in the culture liquid or frozen concentrated bacterial liquid of the propionic acid bacteria.

### Example 4

Relationship between kind (strain) of propionic acid bacteria to be blended (added) into fermented milk substrate or kind (strain) of lactic acid bacterium starter and action of promoting fermentation in fermented milk production

Commercial cow's milk (manufactured by Meiji Co., Ltd.): 77.6 parts by mass, skimmed milk powder (manufactured by Meiji Co., Ltd.): 2.43 parts by mass and water: 17.9 parts by mass were mixed, and the mixture was heated (disinfected) at 95° C. 5 minutes) to prepare raw material milk.

Into this raw material milk, 2.0 parts by mass of a lactic acid bacterium starter (a starter mixture of L. bulgaricus NCIMB 702074 strain and S. thermophilus NCIMB 8510T strain, obtained through arbitrary selection from common strains managed by the depositary institution) and 0.06 parts by mass of each of frozen concentrated bacterial liquids of 7 kinds of propionic acid bacteria, among the 9 kinds of propionic acid bacteria indicated in Table 1 of Example 1, were mixed to prepare fermented milk substrates containing propionic acid bacteria.

The frozen concentrated bacterial liquids of the 7 kinds of propionic acid bacteria used in this Example are frozen concentrates (frozen concentrated bacterial liquids) containing live bacterial cells of the various kinds of propionic acid bacteria, prepared by preparing the medium explained in Example 1, adding (mixing) each of the propionic acid bacteria MEP1404202 strain, ET-3, MEP1404203 strain, MEP1404204 strain, MEP1404205 strain, MEP1404206 strain, and MEP1404208 strain in an amount of 1 part by mass into the medium and statically culturing the propionic acid bacteria (30° C., 3 to 4 days) to prepare culture liquids containing the various kinds of propionic acid bacteria, centrifuging these culture liquids to concentrate the live bacterial cells of the various kinds of propionic acid bacteria 20 times, and then freezing the concentrates in liquid nitrogen (−80° C.).

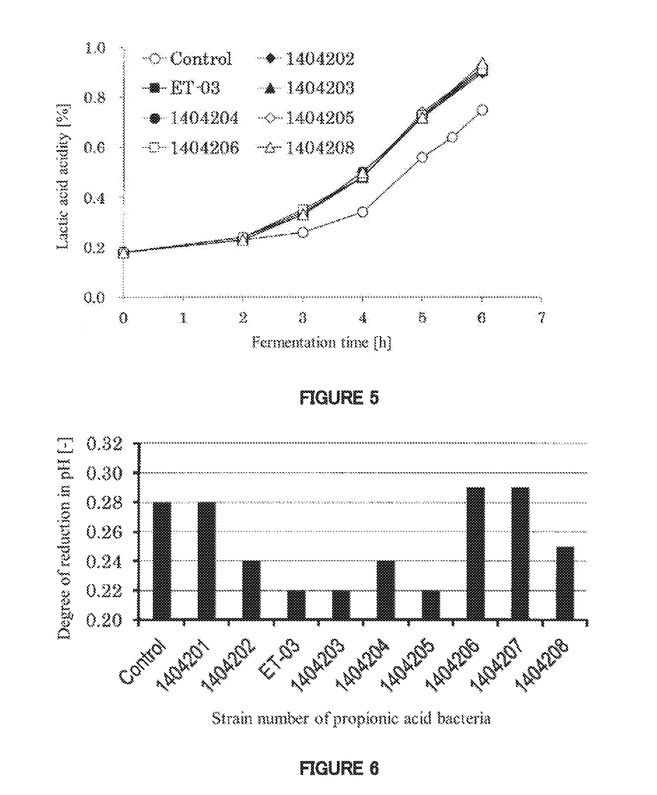

These fermented milk substrates were fermented (43° C.), and the changes in acidity of lactic acid with time are shown in FIG. 5. As a Control, a fermented milk substrate containing no propionic acid bacterium was fermented (43° C.), and the change in acidity of lactic acid with time is shown in FIG. 5.

From FIG. 5, in all of the fermented milk substrates containing the various kinds of propionic acid bacteria, fermentation was promoted, and the time until the acidity reached 0.7% (a guide of the end point of fermentation) was shortened by about 60 minutes as compared with that of the Control (fermented milk substrate containing no propionic acid bacterium).

From this, the lactic acid bacterium starter was not limited to the starter mixture separated from Meiji Bulgaria Yogurt LB81 (trade name), and, even when there was used the starter mixture of L. bulgaricus NCIMB 702074 strain and S. thermophilus NCIMB 8510T strain as a common combination of lactic acid bacteria, the action of promoting fermentation was confirmed in the fermented milk substrates containing the various kinds of propionic acid bacteria.

Accordingly, it was considered that fermentation was promoted in fermented milk substrates containing various kinds of propionic acid bacteria, such as the 7 kinds of propionic acid bacteria among the 9 kinds of strains indicated in Table 1 in Example 1, without limitation on the kind (strain) of the lactic acid bacterium starter.

Next, the relationship between the kind (strain) of propionic acid bacteria to be blended (added) into the fermented milk substrate or the kind (strain) of the lactic acid bacterium starter and the action of suppressing the reduction in pH in the fermented milk was studied.

Commercial cow's milk (manufactured by Meiji Co., Ltd.): 77.6 parts by mass, skimmed milk powder (manufactured by Meiji Co., Ltd.): 2.43 parts by mass and water: 17.9 parts by mass were mixed, and the mixture was heated (disinfected) at 95° C. 5 minutes) to prepare raw material milk.

Into this raw material milk, 2.0 parts by mass of the lactic acid bacterium starter (starter mixture of L. bulgaricus NCIMB 702074 strain and S. thermophilus NCIMB 8510T strain, obtained through arbitrary selection from common strains managed by the depositary institution) and 0.06 parts by mass of each of frozen concentrated bacterial liquids of the 9 kinds of propionic acid bacteria indicated in Table 1 of Example 1 were mixed to prepare fermented milk substrates containing the propionic acid bacteria.

The frozen concentrated bacterial liquids of the 9 kinds of propionic acid bacteria used in this Example are frozen concentrates (frozen concentrated bacterial liquids) containing live bacterial cells of the various kinds of propionic acid bacteria, prepared by preparing the medium explained in Example 1, adding (mixing) each of the propionic acid bacteria MEP1404201 strain, MEP1404202 strain, ET-3, MEP1404203 strain, MEP1404204 strain, MEP1404205 strain, MEP1404206 strain, MEP1404207 strain, and MEP1404208 strain in an amount of 1 part by mass into the medium and statically culturing the propionic acid bacteria (30° C., 3 to 4 days) to prepare culture liquids containing the various kinds of propionic acid bacteria, centrifuging these culture liquids to concentrate the live bacterial cells of the various kinds of propionic acid bacteria 20 times, and then freezing the concentrates in liquid nitrogen (−80° C.).

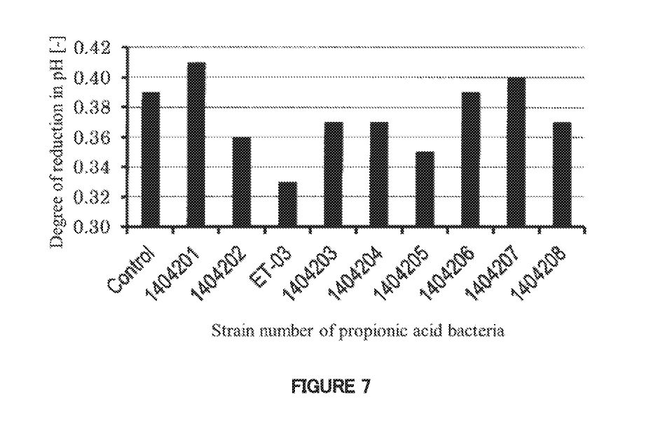

These fermented milk substrates were fermented (43° C.). For the various kinds of fermented milk, the pH of the fresh product immediately after fermentation, the pH of the product refrigerated (5° C.) stored for 7 days immediately after fermentation, and the pH of the product refrigerated (5° C.) stored for 14 days immediately after fermentation were measured to calculate the “(pH of fresh product immediately after fermentation)−(pH of product refrigerated stored for 7 days immediately after fermentation)=(degree of reduction in pH of product refrigerated stored for 7 days immediately after fermentation)” and the “(pH of fresh product immediately after fermentation)−(pH of product refrigerated stored for 14 days immediately after fermentation)=(degree of reduction in pH of product refrigerated stored for 14 days immediately after fermentation).” The calculation results are shown in FIGS. 6 and 7.

As a Control, a fermented milk substrate containing no propionic acid bacterium was fermented (43° C.). Similar calculations were conducted, and the results are shown in FIGS. 6 and 7.

From FIGS. 6 and 7, in the case of the fermented milk produced by blending each of the frozen concentrated bacterial liquids of the various kinds of propionic acid bacteria into the fermented milk substrate, both of the degree of the reduction in pH of the product refrigerated stored for 7 days immediately after fermentation and the degree of the reduction in pH of the product refrigerated stored for 14 days immediately after fermentation were suppressed at an equivalent level or higher, and the action of suppressing the reduction in pH (action of reducing the sourness) was confirmed.

In this way, the action of suppressing the reduction in pH (action of reducing the sourness) was confirmed in the fermented milk substrates containing the various kinds of propionic acid bacteria, without limitation on the kind (strain) of propionic acid bacteria.

1. A method for producing fermented milk, comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.

2. The method for producing fermented milk according to claim 1, wherein a culture liquid of propionic acid bacteria at a range of 0.0001 to 0.25% by mass is blended as the live bacterial cell of propionic acid bacteria into the raw material milk or fermented milk substrate.

3. The method for producing fermented milk according to claim 1, wherein a culture liquid of propionic acid bacteria concentrated to 10 times or more is blended as the live bacterial cell of propionic acid bacteria into the raw material milk or fermented milk substrate.

4. The method for producing fermented milk according to claim 1, wherein the live bacterial cell of propionic acid bacteria at a concentration of 6×10⁶ cfu/mL or more is blended into the raw material milk or fermented milk substrate.

5. A method for shortening the fermentation time in fermented milk production, comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.

6. The method for shortening the fermentation time according to claim 5, wherein a culture liquid of propionic acid bacteria at a range of 0.0001 to 0.25% by mass is blended as the live bacterial cell of propionic acid bacteria into the raw material milk or fermented milk substrate.

7. The method for shortening the fermentation time according to claim 5, wherein a culture liquid of propionic acid bacteria concentrated to 10 times or more is blended as the live bacterial cell of propionic acid bacteria into the raw material milk or fermented milk substrate.

8. The method for shortening the fermentation time according to claim 5, wherein the live bacterial cell of propionic acid bacteria at a concentration of 6×10⁶ cfu/mL or more is blended into the raw material milk or fermented milk substrate.

9. A method for suppressing an increase in sourness of fermented milk, comprising blending a predetermined amount of a live bacterial cell of propionic acid bacteria into raw material milk or a fermented milk substrate immediately before or immediately after blending of a lactic acid bacterium starter into the raw material milk, or simultaneously with blending of the lactic acid bacterium starter into the raw material milk.

10. The method for suppressing an increase in sourness of fermented milk according to claim 9, wherein a culture liquid of propionic acid bacteria at a range of 0.0001 to 0.25% by mass is blended as the live bacterial cell of propionic acid bacteria into the raw material milk or fermented milk substrate.

11. The method for suppressing an increase in sourness of fermented milk according to claim 9, wherein a culture liquid of propionic acid bacteria concentrated to 10 times or more is blended as the live bacterial cell of propionic acid bacteria into the raw material milk or fermented milk substrate.

12. The method for suppressing an increase in sourness of fermented milk according to claim 9, wherein the live bacterial cell of propionic acid bacteria at a concentration of 6×10⁶ cfu/mL or more is blended into the raw material milk or fermented milk substrate.
